(19) United States
(12) Patent Application Publication
(10) Pub. No.: US 2007/0123729 A1
(43) Pub. Date: May 31, 2007

(54) Synthesis of trithiocarbonates

(76) Inventor: William Brown Farnham, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/289,108

(22) Filed: Nov. 29, 2005

(51) Int. Cl. C07C 329/00

(52) U.S. Cl. 558/244

(57) ABSTRACT

This invention provides an efficient method for synthesizing trithiocarbonates, RSC(S)SR′ that can be used, for example, as RAFT agents in the living polymerization of styrenics, acrylates, and other olefinic monomers.

### FIELD OF THE INVENTION

This invention provides an efficient method for synthesizing trithiocarbonates, RSC(S)SR′. Such compounds can be used, for example, as RAFT agents in the living polymerization of styrenics, acrylates, and other olefinic monomers.

### BACKGROUND

RAFT (reversible addition fragmentation chain transfer) processes have been disclosed for the preparation of low-polydispersity polymers from styrenics, acrylates, methacrylates, and copolymers thereof, see e.g., WO 99/05099, WO 99/31144 and EP0910587.

Trithiocarbonates have been identified as suitable RAFT agents, but commercially attractive methods for their preparation have been lacking. For example, methods that convert a thiol to the corresponding sodium salt frequently involve the use of sodium hydride, a potentially hazardous chemical when handled at large scales. Previous methods have used multistep reactions involving isolation and purification steps difficult to perform at larger scale. Previous methods have also reported low yields.

A continuing need exists for an efficient and scalable process for RAFT agents.

### SUMMARY

One embodiment of this invention provides a process for preparing a trithiocarbonate having the formula RSC(S)SR′, wherein R is a substituted or unsubstituted C₁-C₂₀ linear or branched alkyl group, a substituted or unsubstituted C₃-C₆ cyclic alkyl group, a substituted or unsubstituted aryl group, or a heterocyclic group and wherein R′ is a substituted or unsubstituted C₁-C₁₀ linear or branched alkyl group or a substituted or unsubstituted C₃-C₆ cyclic alkyl group, the process comprising reacting a thiol having the formula RSH, sequentially with an aqueous base, carbon disulfide and an alkylating agent having the formula R′X, wherein X is chlorine, bromine or iodine, in a water/alcohol solvent medium, whereby a mixture comprising the product RSC(S)SR′ and a by-product salt of chlorine, bromine or iodine are formed in the solvent medium.

### DETAILED DESCRIPTION

The process of this invention provides a practical, single step, high yielding procedure for the preparation of a wide variety of useful trithiocarbonates having the formula RSC(S)SR′. In one embodiment of this invention, a thiol, RSH, is reacted sequentially with an aqueous base and carbon disulfide and an alkylating agent having the formula R′X in a solvent mixture comprising about 1-20 vol % water and about 80-99 vol % alcohol.

Suitable alkyl thiols, RSH, are those in which R is a substituted or unsubstituted C₁-C₂₀ linear or branched alkyl group, a substituted or unsubstituted C₃-C₆ cyclic alkyl group, a substituted or unsubstituted aryl group, or a heterocyclic group. Suitable substituents include alkyl, halo, cyano, aryl, and alkoxy groups. Suitable R′ groups include substituted or unsubstituted C₁-C₁₀ linear or branched alkyl groups and substituted or unsubstituted C₃-C₆ cyclic alkyl groups. Suitable substituents include alkyl, halo, cyano, aryl, alkoxy, and carboxylate groups. X is chlorine, bromine or iodine.

During the course of the reaction, the halide from R′X is converted to a halide salt as a by-product. In one embodiment, the trithiocarbonate product can then be isolated by filtering the reaction mixture to remove the halide by-product salt and recovering the product from the filtrate to provide pure, e.g. >98% purity, crystals of the product.

Solubility of the product, RSC(S)SR′, is influenced by the selection of R and R′, and by the temperature and composition of the aqueous alcohol medium. In some embodiments of this invention, it may be convenient to select R and R′ such that the product is soluble in the initial aqueous alcohol medium to allow separation from the by-product salt by filtration, but insoluble in an altered solvent medium. As illustrated in the Example, crystallization of the desired product from the aqueous alcohol can be induced by increasing the water content of the solvent and decreasing the temperature.

The aqueous base can be any alkali metal hydroxide, but sodium hydroxide and potassium hydroxide are preferred.

In one preferred embodiment of the invention, the aqueous base is sodium hydroxide, RSH is dodecanethiol and R′X is chloroacetonitrile as shown in the reaction below. The preferred alcohol for this reaction is isopropanol. The reaction provides the intermediate RSC(S)SNa which is then treated with chloroacetonitrile to form the trithiocarbonate RAFT agent, as shown:
C₁₂H₂₅SH+NaOH+CS₂→[C₁₂H₂₅SCS₂Na]+ClCH₂CN→C₁₂H₂₅SC(S)SCH₂CN+NaCl
Following the reaction, the halide salt, NaCl, is removed by filtration. The product C₁₂H₂₅SC(S)SCH₂CN is recovered at reduced temperatures from the filtrate medium on dilution with water as a crystalline material. One advantage of this embodiment is the easy removal of the halide salt by filtration and the crystallization of the product from the same medium after a modest increase in water content, giving high yields and highly pure product. In the event the trithiocarbonate product with a different combination of R and R′ is not crystalline; the product can be recovered by a phase separation upon water addition.

The reaction can be performed at a temperature of from about −10° C. to about 40° C. Preferably the temperature is about −5° C. to about 15° C. Most preferably, the temperature is about 0° C. to about 5° C.

In one embodiment, the aqueous alcohol is a water/alcohol mixture with a ratio of 1-20 vol % water and 80-99 vol % alcohol. Preferably the range is 3-10 vol % water and 90-97 vol % alcohol. More preferably, the ratio is 4-6 vol % water and 94-96 vol % alcohol.

The alcohol is a water-soluble alcohol or a mixture of water-soluble alcohols. In one embodiment, the alcohol is isopropanol.

Many thiols, RSH, and halo compounds, R′X, useful in this invention are commercially available. Others can be readily synthesized by techniques known in the art.

### EXAMPLE

This example demonstrates the preparation of S-cyanomethyl S-dodecyl trithiocarbonate RAFT agent: A 1000 mL 3-neck round bottom flask (fitted with mechanical stirrer, septum, thermocouple well, and reflux condenser with N₂ bubbler) was charged with sodium hydroxide (12.18 g, 304.5 mmol) and water (30 mL). Isopropanol (500 mL) was added to the solution of sodium hydroxide, and the reaction mixture was cooled to about 5° C. The cooled reaction mixture was treated dropwise with dodecanethiol (60.6 g, 300 mmol).

The reaction mixture was stirred for 30 min at 5° C., cooled to 0° C., and treated with carbon disulfide (24.0 g, 315 mmol) by syringe over a ca. 10 min period to produce a yellow solution which was stirred at ca. 0°-5° C. for 0.5 h.

The reaction mixture was treated with chloroacetonitrile (23.8 g, 315 mmol) dropwise by syringe over a ca. 20 min period while maintaining the temperature between 0 and 5° C. The reaction mixture was stirred at 0° C. for 2 h.

The reaction mixture was warmed to ca. 30° C. and filtered to remove sodium chloride. The solid residue was washed with a 5 mL portion of isopropanol. The combined filtrate was treated with water (50 mL). Product crystals of S-cyanomethyl S-dodecyl trithiocarbonate were formed as the temperature decreased. After the bulk of crystals had formed at room temperature, the mixture was cooled to 0° C. for 1.5 h and the first crop of crystals was collected. There was obtained 82.3 g of bright yellow flakes after drying. The filtrate was treated with another 50 mL water and chilled to provide 8.5 g of the product in the second crop of crystals. ¹H NMR analyses showed both crops of ca. 98% purity (93% isolated yield).

What is claimed is:

1. A process for preparing a trithiocarbonate having the formula RSC(S)SR′, wherein R is a substituted or unsubstituted C₁-C₂₀ linear or branched alkyl group, a substituted or unsubstituted C₃-C₆ cyclic alkyl group, a substituted or unsubstituted aryl group, or a heterocyclic group and wherein R′ is a substituted or unsubstituted C₁-C₁₀ linear or branched alkyl group or a substituted or unsubstituted C₃-C₆ cyclic alkyl group, the process comprising reacting a thiol having the formula RSH, sequentially with an aqueous base, carbon disulfide and an alkylating agent having the formula R′X, wherein X is chlorine, bromine or iodine, in a water/alcohol solvent medium, whereby a mixture comprising the product RSC(S)SR′ and a by-product salt of chlorine, bromine or iodine are formed in the solvent medium.

2. The process of claim 1, wherein the substituent in the substituted alkyl or aryl group of R is selected from a group comprising alkyl, halo, cyano, aryl, and alkoxy groups and the substituent in the substituted alkyl group of R′ is selected from a group comprising alkyl, halo, cyano, aryl, alkoxy, and carboxylate groups.

3. The process of claim 1, wherein the aqueous base is sodium hydroxide or potassium hydroxide.

4. The process of claim 1, wherein X is chlorine.

5. The process of claim 1, wherein the water/alcohol solvent medium is 1-20 vol % water and 80-99 vol % alcohol.

6. The process of claim 5, wherein the water/alcohol solvent medium is 3-10 vol % water and 90-97 vol % alcohol.

7. The process of claim 1, wherein the alcohol is isopropanol.

8. The process of claim 5, wherein the alcohol is isopropanol.

9. The process of claim 1, wherein the temperature of the reaction is about −10° C. to about 40° C.

10. The process of claim 9, wherein the temperature of the reaction is about −5° C. to about 15° C.

11. The process of claim 1, further comprising filtering the mixture to remove the by-product salt.

12. The process of claim 11, further comprising recovering the trithiocarbonate RSC(S)SR′ product from the solvent medium.

13. The process of claim 12, wherein the product RSC(S)SR′ is recovered by crystallization from the solvent medium.

14. The process of claim 1, wherein the aqueous base is sodium hydroxide, R is C₁₂H₂₅, R′X is chloroacetonitrile, the alcohol is isopropanol and the product is S-cyanomethyl S-dodecyl trithiocarbonate.

15. The process of claim 14, further comprising filtering the mixture to remove the by-product salt and recovering the product, S-cyanomethyl S-dodecyl trithiocarbonate, by crystallization from the solvent medium.
